(19) United States
(12) Patent Application Publication
(10) Pub. No.: US 2023/0037947 A1
(43) Pub. Date: Feb. 9, 2023

(54) DETECTION OF MISMATCHED SENSORS

(71) Applicant: Covidien LP, Mansfield, MA (US)

(72) Inventors: Rebecca J.S. Haag, Broomfield, CO (US); Yoana Lizbet Arellano, Denver, CO (US); Gregory J. Lund, Boulder, CO (US); Joseph Stoskus, Parker, CO (US); Jeffrey Booker, Brighton, CO (US); Prajakta Kulkarni, Broomfield, CO (US); Robert W. Eikel, Lafayette, CO (US); John A. Battista, JR., Lafayette, CO (US)

(21) Appl. No.: 17/816,661

(22) Filed: Aug. 1, 2022

Related U.S. Application Data
(60) Provisional application No. 63/228,969, filed on Aug. 3, 2021.

(51) Int. Cl. A61B 5/00; G08B 21/18

(52) U.S. Cl. CPC A61B 5/7221; G08B 21/18; A61B 2560/0276

(57) ABSTRACT

In examples described herein, a patient monitoring device is configured to determine, based on signals received from signal acquisition devices, whether there is a mismatch among sensors connected to the signal acquisition devices. In some examples, each signal acquisition device is configured to communicate to the monitoring device what types of sensors are attached to the respective signal acquisition device. Additionally or alternatively, each signal acquisition device can determine whether there is a mismatch among the sensors connected to the signal acquisition device and send an indication of the match or mismatch to the monitoring device.

This application claims the benefit of U.S. Provisional Application No. 63/228,969, filed Aug. 3, 2021, and entitled, “DETECTION OF MISMATCHED SENSORS,” the entire content of which is incorporated by reference herein.

### TECHNICAL FIELD

This disclosure relates to patient monitoring.

### BACKGROUND

A physiological monitor device can determine and present physiological parameter values of a subject based on signals from a signal acquisition device. The signal acquisition device may, for example, receive and process raw analog signals from a sensor that is attached to the subject. For example, the signal acquisition device may include circuitry configured to convert the raw analog signals to digital values and/or circuitry for determining physiological parameter values based on the received signals. The physiological monitor device can present the physiological parameter values that it receives from the signal acquisition device.

### SUMMARY

The present disclosure describes devices, systems, and methods for detecting a mismatch of sensors coupled to a patient monitoring device. A monitoring device can be configured for use with multiple types of sensors. For example, the sensors can be configured to sense the same physiological parameter (e.g., blood pressure, blood oxygen saturation, regional oxygen saturation, or the like), but each type of sensor can be configured for a particular a type of subject (e.g., age) or a location on the subject. For one or more reasons, it may be desirable in some cases to connect sensors that are all of the same type to the monitoring device. For example, the monitoring device may use different algorithms or the like based on the type of sensors that are coupled to the monitoring device. As an example, different sensor types may be associated with different algorithms for determining whether a sensor is making good contact with tissue of the subject.

In examples described herein, a monitoring device is configured to determine, based on the signals received from signal acquisition devices, whether there is a mismatch among any of the sensors. In some examples described herein, each signal acquisition device is configured to communicate to the monitoring device what types of sensors are attached to the respective signal acquisition device. Additionally or alternatively, each signal acquisition device can determine whether there is a mismatch among the sensors connected to the signal acquisition device and send an indication of the match or mismatch to the physiological monitoring device.

The details of one or more examples are set forth in the accompanying drawings and the description below. Other features, objects, and advantages will be apparent from the description and drawings, and from the claims.

### BRIEF DESCRIPTION OF THE DRAWINGS

FIG. 1 is a conceptual block diagram illustrating an example device.

FIG. 2 is a diagram illustrating an example of sensors of a first type connected to a monitoring device via signal acquisition devices.

FIG. 3 is a diagram illustrating an example of sensors of a second type connected to a monitoring device via signal acquisition devices.

FIG. 4 is a conceptual diagram illustrating an example graphical user interface including an alert presented on a display.

FIGS. 5-7 are flow diagrams illustrating example techniques for determining a sensor mismatch, in accordance with techniques described herein.

### DETAILED DESCRIPTION

A physiological monitoring device may be configured to determine physiological parameter values based on signals received from signal acquisition devices that are connected to sensors attached to a subject. There may be multiple types of sensors that can be used with a particular device, where each sensor is specialized for a type of subject (e.g., age) or a location on the subject. Thus, it may be desirable in some cases to use sensors that are all of the same type, for example, in order to avoid having an adult sensor and a pediatric sensor attached to the same subject.

Different sensors may allow for different functionality for the physiological monitoring device. For example, each sensor type may be associated with an algorithm applied by the monitoring device to the data received from a signal acquisition device. The monitoring device may be configured to implement the different algorithms by, for example, applying an offset (e.g., absolute or relative) to the data received from the signal acquisition devices. In response to determining that all of the sensors are of the same type, the monitoring device may be configured to apply, to the physiological parameter data, the algorithm associated with that type of sensor.

Additionally or alternatively, different sensor types may be associated with different algorithms for determining whether a sensor is making good contact with tissue of the subject. For example, for a first type of sensor, the monitoring device may be configured to cause the sensor to emit a first and a second wavelength of light for testing whether the sensor is making good contact. The monitoring device may be configured to also cause the first type of sensor to emit a third and a fourth wavelength of light for sensing a physiological parameter of subject. For a second type of sensor, the monitoring device may be configured to cause the sensor to emit the third and fourth wavelengths for sensing the physiological parameter of subject. The second type of sensor may not have the ability to emit the first and second wavelengths of light.

In order to detect a sensor mismatch, each signal acquisition device may be configured to communicate to the monitoring device what types of sensors are attached to the respective signal acquisition device. Additionally or alternatively, each signal acquisition device can determine whether there is a mismatch among the sensors connected to the signal acquisition device and send an indication of the match or mismatch to the physiological monitoring device. The indication may be as simple as a single bit or field indicating a match or mismatch, or the indication may include additional data such the type and location of each sensor. The monitoring device can determine, based on the signals received from the signal acquisition devices, whether there is a mismatch among any of the sensors. In some examples, the monitoring device can perform this sensor check and, in the event of a sensor match, select an algorithm without any user input.

The monitoring device may be configured to output an indication of the mismatched sensors to a clinician. The monitoring device may be configured to inform the clinician of which sensor is mismatched and the type of mismatched sensor that is being used. The monitoring device may command the signal acquisition device to enter a standby mode in response to detecting the mismatched sensor. By putting the signal acquisition devices in a standby mode, the monitoring device may prevent incorrect data from being presented to the clinician. By implementing these techniques, the monitoring device may also ensure the safety of the subject by reducing the likelihood that the physiological data is incorrectly calculated due to using the wrong algorithm.

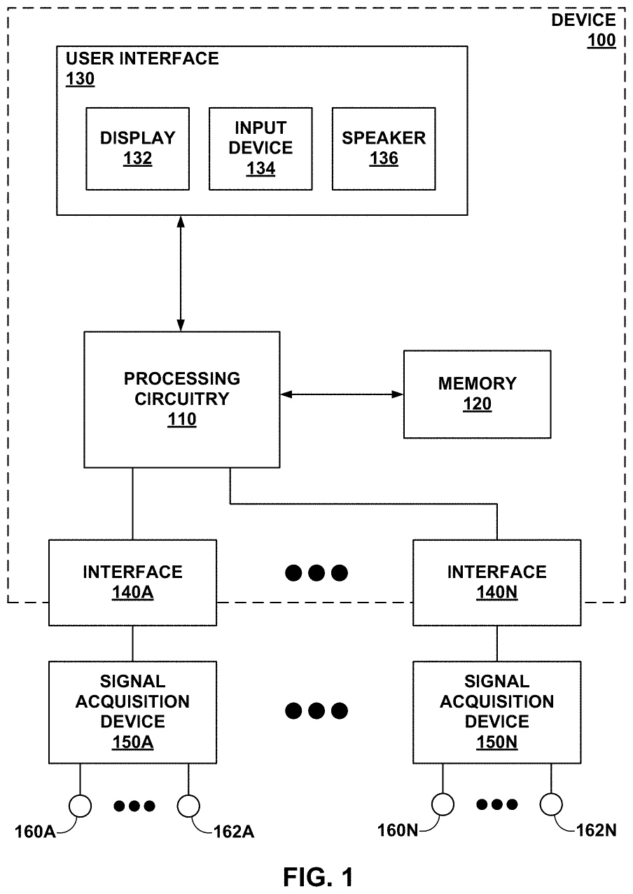

FIG. 1 is a conceptual block diagram illustrating an example device 100. While this disclosure describes an example of device 100 as a regional oximetry device, techniques described herein can also be used for other physiological monitoring devices. Device 100 includes processing circuitry 110, memory 120, user interface 130, display 132, input device 134, speaker 136, and interfaces 140A-140N. Signal acquisition devices 150A-150N and sensors 160A-162N shown in FIG. 1 as external to device 100, but signal acquisition devices 150A-150N and/or sensors 160A-162N may be integrated into device 100 in some examples.

In some examples, device 100 may be configured to determine and display the cerebral autoregulation status of a patient, e.g., during a medical procedure or for more long-term monitoring, such as monitoring of prenatal infants, children, or adults. A clinician may receive information regarding the cerebral autoregulation status of a patient via display 132 and speaker 136 and adjust treatment or therapy to the patient based on the cerebral autoregulation status information. Although device 100 is described as an example device herein, other devices may calculate blood pressure and/or use blood pressure for other physiological monitoring and perform similar a compensation process on blood pressures subjected to abrupt changes in the measured blood pressure values.

Processing circuitry 110 as well as other processors, processing circuitry, controllers, control circuitry, and the like, described herein, may include one or more processors. Processing circuitry 110 may include any combination of integrated circuitry, discrete logic circuitry, analog circuitry, such as one or more microprocessors, digital signal processors (DSPs), application specific integrated circuits (ASICs), or field-programmable gate arrays (FPGAs). In some examples, processing circuitry 110 may include multiple components, such as any combination of one or more microprocessors, one or more DSPs, one or more ASICs, or one or more FPGAs, as well as other discrete or integrated logic circuitry, and/or analog circuitry.

Memory 120 may be configured to store data relating to the types of sensors 160A-160N and 162A-162N. Memory 120 may be configured to also store algorithms for use with each sensor type, including offset values and on/off-tissue algorithms for each sensor type. Memory 120 can also store wavelengths of light for using in measuring physiological parameters and performing on/off-tissue measurements. Memory 120 may be configured to store measurements of any physiological parameters such as blood pressure, oxygen saturation, blood volume, and other physiological parameters, relationships between blood pressure and physiological parameters, MAP values, rSO2 values, COx values, BVS values, HVx values, and/or value(s) of a lower limit of autoregulation (LLA) and/or an upper limit of autoregulation (ULA), for example.

Memory 120 may store program instructions, which may include one or more program modules, which are executable by processing circuitry 110. When executed by processing circuitry 110, such program instructions may cause processing circuitry 110 to provide the functionality ascribed to it herein. For example, memory 120 may store instructions regarding how to determine a match or mismatch among sensors 160A-160N and 162A-162N. The program instructions may be embodied in software, firmware, and/or RAMware. Memory 120, as well as other memory devices described herein, may include any volatile, non-volatile, magnetic, optical, circuitry, or electrical media, such as a random access memory (RAM), read-only memory (ROM), non-volatile RAM (NVRAM), electrically-erasable programmable ROM (EEPROM), flash memory, or any other digital media.

User interface 130, display 132, and/or speaker 136 may be configured to present information to a user (e.g., a clinician). User interface 130 and/or display 132 may be configured to present a graphical user interface to a user, where each graphical user interface may include indications of sensors 160A-160N and 162A-162N. For example, processing circuitry 110 may be configured to present an indication of a match or mismatch among sensors 160A-160N and 162A-162N. In some examples, in response to determining there is a mismatch among sensors 160A-160N and 162A-162N, processing circuitry 110 may be configured to present a notification (e.g., an alert) indicating the mismatch such as indications of each of the types of sensors 160A-160N and 162A-162N. As another example, processing circuitry 110 may be configured to present, via display 132, estimates of one or more physiological parameters such as the regional oxygen saturation (rSO2), the blood oxygen saturation (SpO2), pulse rate information, respiration rate information, blood pressure, any other patient parameters, or any combination thereof.

User interface 130 and/or display 132 may include a monitor, cathode ray tube display, a flat panel display such as a liquid crystal (LCD) display, a plasma display, or a light emitting diode (LED) display, personal digital assistant, mobile phone, tablet computer, laptop computer, any other suitable display device, or any combination thereof. User interface 130 may also include means for projecting audio to a user, such as speaker(s). Processing circuitry 110 may be configured to present, via user interface 130, a visual, audible, or somatosensory notification (e.g., an alarm signal) indicative of the patient's autoregulation status. User interface 130 may include or be part of any suitable device for conveying such information, including a computer workstation, a server, a desktop, a notebook, a laptop, a handheld computer, a mobile device, or the like. In some examples, processing circuitry 110 and user interface 130 may be part of the same device or supported within one housing (e.g., a computer or monitor).

Input device 134 may include one or more of any type of user input device such as a keyboard, a mouse, a touch screen, buttons, switches, a microphone, a joystick, a touch pad, or any other suitable input device or combination of input devices. In other examples, input device 134 may be a pressure-sensitive or presence-sensitive display that is included as part of display 132. In some examples, processing circuitry 110 may determine the type of sensor that the user is attempting to use based on user inputs received by input device 134. In some examples, user interface 130 includes speaker 136 that is configured to generate and provide an audible sound that may be used in various examples, such as for example, sounding an audible notification in the event that processing circuitry 110 determines a sensor mismatch.

Interfaces 140A-140N may be configured to connect processing circuitry 110 to signal acquisition devices 150A-150N. Interfaces 140A-140N may include a pinout that matches a pinout of signal acquisition devices 150A-150N. In some examples, device 100 may be configured to supply electrical power to signal acquisition devices 150A-150N and sensors 160A-160N and 162A-162N through interfaces 140A-140N.

Although not shown in FIG. 1, an adaptor cable or other intermediate device may be connected between one of interfaces 140A-140N and one of signal acquisition devices 150A-150N or between one of signal acquisition devices 150A-150N and one of sensors 160A-160N and 162A-162N. FIG. 1 depicts two sensors connected to each signal acquisition device and two signal acquisition devices connected to device 100, but other numbers of sensors may be connected to each signal acquisition device and other numbers of signal acquisition devices may be connected to device 100 in some examples. Additional example details of sensors for oxygen saturation are described in commonly assigned U.S. Pat. No. 10,849,538, entitled “Sensor Verification Through Forward Voltage Measurements,” issued on Dec. 1, 2020, the entire contents of which are incorporated herein by reference.

Signal acquisition devices 150A-150N may be configured to receive physiological signals sensed by respective sensors 160A-160N and 162A-162N and communicate the physiological signals to processing circuitry 110 through interfaces 140A-140N. Sensors 160A-160N and 162A-162N may include any sensing hardware configured to sense a physiological parameter of a patient, such as, but not limited to, one or more electrodes, optical receivers, blood pressure cuffs, or the like. Signal acquisition devices 150A-150N may convert the physiological signals to usable signals for processing circuitry 110, such as digital values associated with the physiological parameters. Signal acquisition device 150A may determine, based on the types of sensors 160A-162A, which algorithm to apply to the raw signals sensed by sensors 160A-162A.

Signal acquisition devices 150A-150N may receive signals indicating physiological parameters from a patient, such as, but not limited to, blood pressure, regional oxygen saturation, heart rate, and respiration. Signal acquisition devices 150A-150N may include, but are not limited to, blood pressure sensing circuitry, oxygen saturation sensing circuitry, heart rate sensing circuitry, temperature sensing circuitry, electrocardiography (ECG) sensing circuitry, electroencephalogram (EEG) sensing circuitry, or any combination thereof. In some examples, signal acquisition devices 150A-150N and/or processing circuitry 110 may include signal processing circuitry such as an analog-to-digital converter.

Each of signal acquisition devices 150A-150N may include circuitry configured to receive raw sensed signals from respective sensors 160A-160N and 162A-162N. Each of signal acquisition devices 150A-150N may include an analog-to-digital converter (ADC) configured to generate digital values based on the raw signals. For example, each of signal acquisition devices 150A-150N may be configured to determine physiological parameter values based on the raw signals received from sensors 160A-160N and 162A-162N. Signal acquisition devices 150A-150N can send the physiological parameter values to device 100 through interfaces 140A-140N.

Each of signal acquisition devices 150A-150N may be configured to determine the type of each sensor that is attached to the respective signal acquisition device. Each of signal acquisition devices 150A-150N may be configured to determine whether there is a match or mismatch among the sensor types attached to the respective signal acquisition device. Each of signal acquisition devices 150A-150N may be configured to send data to device relating to the sensor types and/or a match or mismatch between the sensor types. For example, signal acquisition device 150A can determine the type of sensor 160A and the type of sensor 162A because of each of sensors 160A and 162A may send an identifier signal to signal acquisition device 150A. Signal acquisition device 150A may communicate with sensor 160A to retrieve an identification of sensor 160A, which may be from a memory, a pin connection, or a resistor value. Signal acquisition device 150A may be configured to determine whether the type of sensor 160A matches the type of sensor 162A. Signal acquisition device 150A may then send to device 100 an indication (e.g., a flag) of the match or mismatch between sensors 160A and 162A. Additionally or alternatively, signal acquisition device 150A can send indications of the type of sensors 160A and 162A to device 100.

Processing circuitry 110 may receive physiological signals and/or physiological parameter values from signal acquisition devices 150A-150N. Processing circuitry 110 may also receive indications of the types of sensors 160A-160N and 162A-162N and/or indications of whether each of signal acquisition devices 150A-150N has determined a match or mismatch among the respective sensors connected to the respective signal acquisition device. Processing circuitry 110 may be configured to whether any of signal acquisition devices 150A-150N has indicated a mismatch. Processing circuitry 110 may be configured to also determine whether all of sensors 160A-160N and 162A-162N are of the same type.

In response to determining a mismatch reported by one of signal acquisition devices 150A-150N or a mismatch determined by processing circuitry 110, processing circuitry 110 may be configured to generate an alert. Processing circuitry 110 can output the alert by user interface 130 or to another device. Additionally or alternatively, processing circuitry 110 may be configured to send a command to signal acquisition devices 150A-150N to, for example, enter a standby mode. Processing circuitry 110 may be configured to record occurrences of mismatched sensors to the patient trend data stored in memory 120.

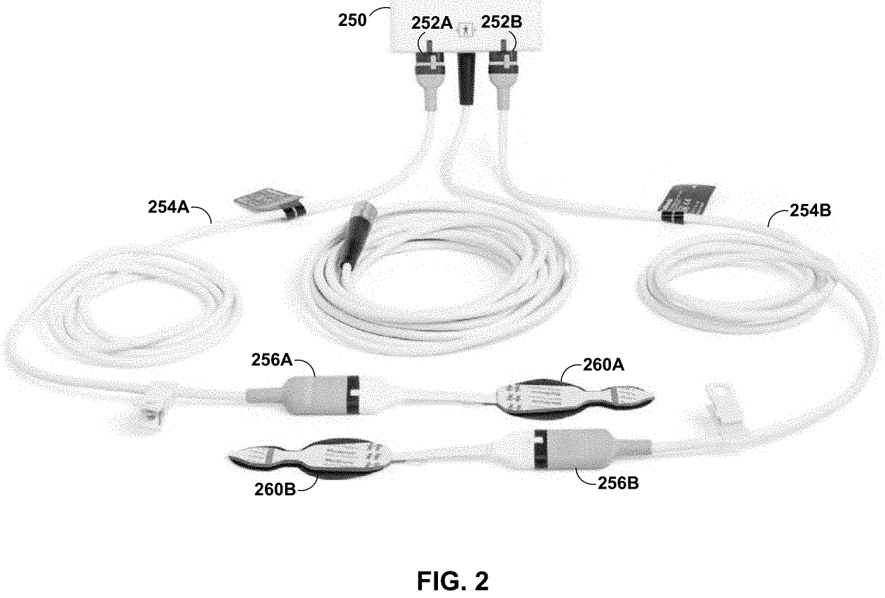

FIG. 2 is a diagram illustrating an example of sensors 260A and 260B of a first type connected to a monitoring device via signal acquisition device 250 and extension cables 254A and 254B. FIG. 2 depicts sensor 260A connected to signal acquisition device 250 via interfaces 252A and 256A, and extension cable 254A. FIG. 2 also depicts sensor 260B connected to signal acquisition device 250 via interfaces 252B and 256B, and extension cable 254B. The type of sensor 260A may be the same as the type of sensor 260B. For example, sensors 260A and 260B may be pediatric-type sensors. Therefore, signal acquisition device 250 may determine that sensor 260A matches sensor 260B.

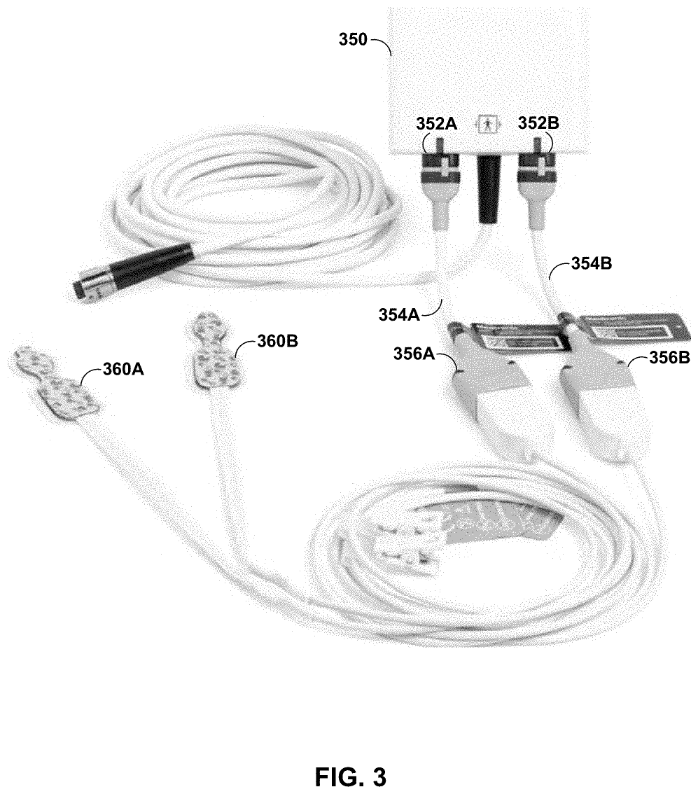

FIG. 3 is a diagram illustrating an example of sensors of a second type connected to a monitoring device via signal acquisition devices. FIG. 3 depicts sensor 360A connected to signal acquisition device 350 via interfaces 352A and 356A and adaptor cable 354A. FIG. 3 also depicts sensor 360B connected to signal acquisition device 350 via interfaces 352B and 356B and adaptor cable 354B. Adaptor cables 354A and 354B may be used as intermediary components where sensors 360A and 360B cannot connect directly to signal acquisition device 350. The type of sensor 360A may be the same as the type of sensor 360B. For example, sensors 360A and 360B may be infant-type sensors. Therefore, signal acquisition device 350 may determine that sensor 360A matches sensor 360B.

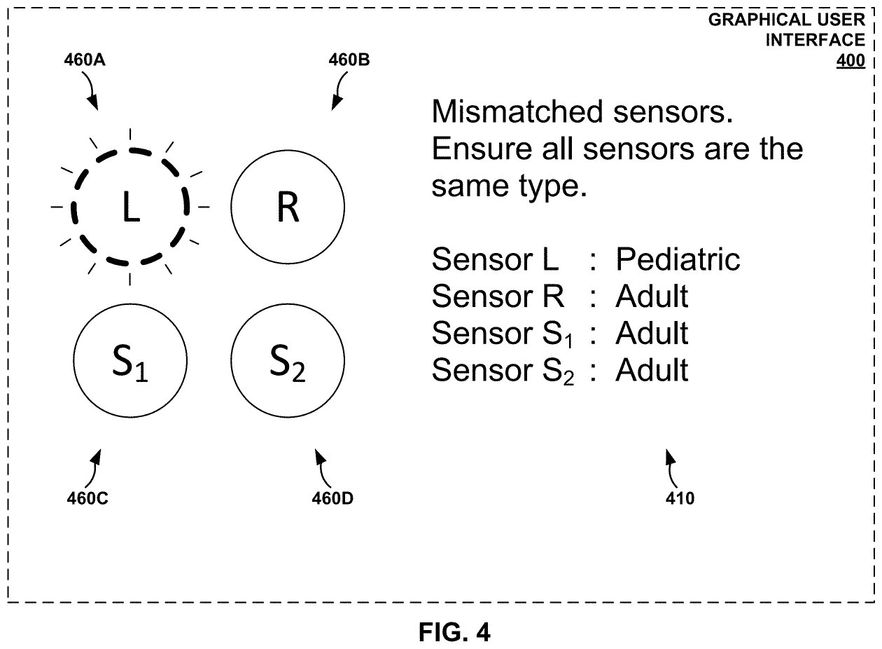

FIG. 4 is a conceptual diagram illustrating an example graphical user interface 400 including an alert presented on a display. Graphical user interface 400 includes text block 410 and graphical icons 460A-460D. Graphical icons 460A-460D indicate the sensors connected to the monitoring device. Processing circuitry of the monitoring device can illuminate graphical icon 460A in response to determining that the type of the sensor associated with graphical icon 460A (e.g., pediatric) is different from the type of the sensors associated with graphical icons 460B-460D (e.g., adult). The processing circuitry may be configured to also present text block 410 in graphical user interface 400 to explain the sensor mismatch.

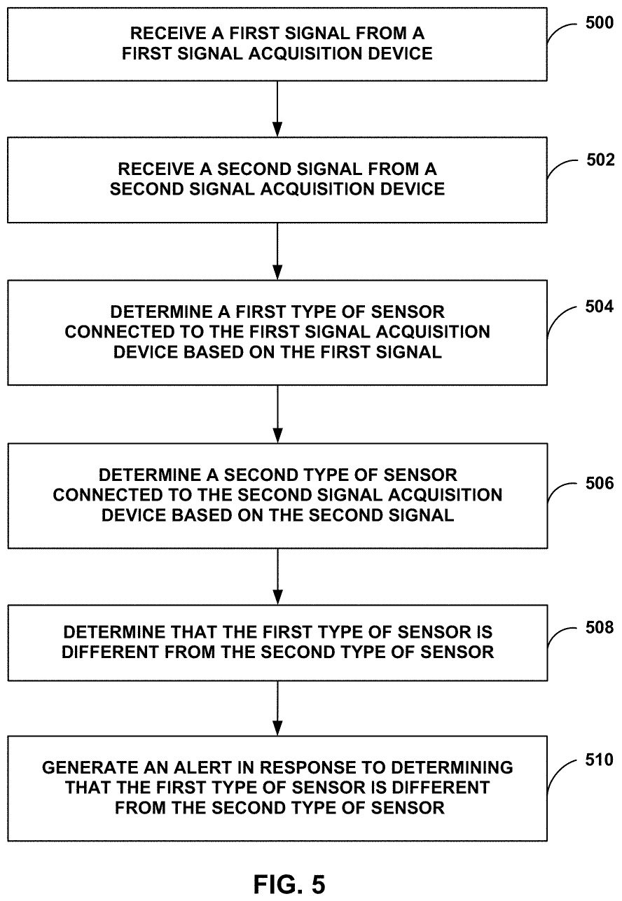

FIGS. 5-7 flow diagrams illustrating example techniques for determining a sensor mismatch, in accordance with techniques described herein. Although FIGS. 5-7 are described with respect to device 100 (FIG. 1), in other examples, other devices may perform any part of the techniques of FIGS. 5-7.

In the example of FIG. 5, processing circuitry 110 receives a first signal from signal acquisition device 150A (500). The first signal may include data such as the types of sensors 160A-162A, whether there is a mismatch among sensors 160A-162A, and/or physiological parameter values sensed by sensors 160A-162A. Processing circuitry 110 also receives a second signal from signal acquisition device 150N (502). The second signal may include data such as the types of sensors 160N-162N, whether there is a mismatch among sensors 160N-162N, and/or physiological parameter values sensed by sensors 160A-162N.

In the example of FIG. 5, processing circuitry 110 determines a first type of sensor 160A connected to signal acquisition device 150A based on the first signal (504). Processing circuitry 110 also determines a second type of sensor 160N connected to signal acquisition device 150N based on the second signal (506). In some examples, processing circuitry 110 may be configured to determine the types of all of sensors 160A-162A and 160N-162N based on the first and second signals. Processing circuitry 110 then determines that the first type of sensor 160A is different from the second type of sensor 160N (508). For example, processing circuitry 110 may determine that sensor 160A is an adult sensor, and processing circuitry 110 may determine that sensor 160N is a pediatric sensor.

In the example of FIG. 5, responsive to determining that the first type of sensor 160A is different from the second type of sensor 160N, processing circuitry 110 may be configured to generate an alert (510). To generate the alert, processing circuitry 110 may be configured to output an indication of the mismatch between sensors 160A and 160N via display 132 and/or speaker 136. Additionally or alternatively, to generate the alert, processing circuitry 110 may be configured to refrain from presenting physiological parameter values received from signal acquisition devices 150A-150N. In some examples, to generate the alert, processing circuitry 110 may be configured to command signal acquisition devices 150A-150N to refrain from sending physiological parameter values to device 100 and/or to command signal acquisition devices 150A-150N to enter a standby mode.

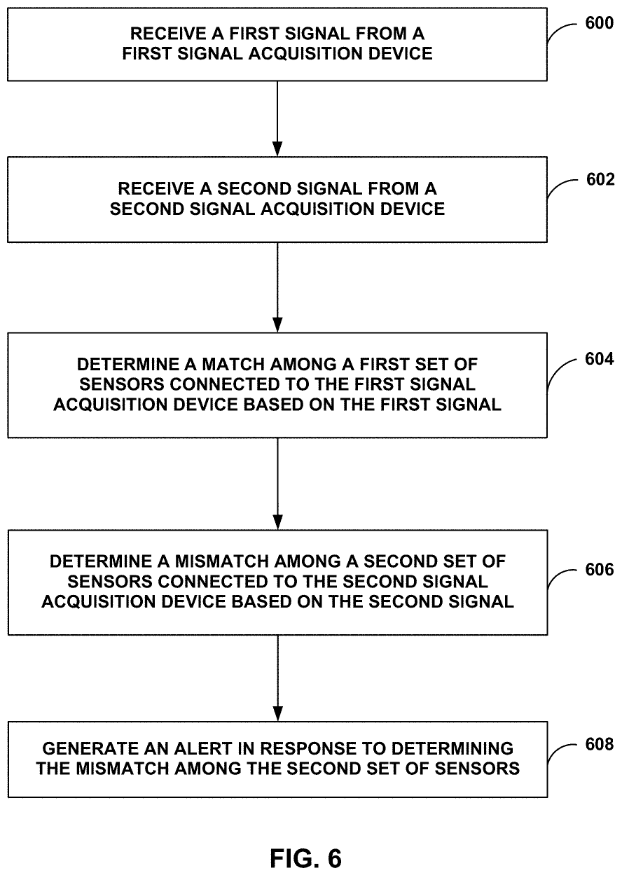

In the example of FIG. 6, processing circuitry 110 receives first signal from signal acquisition device 150A (600) and receives a second signal from signal acquisition device 150N (602). The first and second signals may include data such as the types of sensors 160N-162N, whether there is a mismatch among sensors 160N-162N, and/or physiological parameter values sensed by sensors 160A-162N. Processing circuitry 110 determines a match among sensors 160A-162A based on the first signal (604). For example, processing circuitry 110 may determine that each of sensors 160A-162A is the same type.

In the example of FIG. 6, processing circuitry 110 determines a mismatch among sensors 160N-162N based on the second signal (606). For example, processing circuitry 110 may determine that sensor 160N is a different type of sensor than sensor 162N. Processing circuitry 110 generates an alert in response to determining the mismatch among sensors 160N-162N. For example, to generate the alert, processing circuitry 110 may be configured to refrain presenting physiological parameter values from all of signal acquisition devices 150A-150N in response to determining that just one of signal acquisition devices 150A-150N has a mismatch. To generate the alert, processing circuitry 110 may be configured to command all of signal acquisition devices 150A-150N to refrain from sending physiological parameter values to device 100 and/or to command all of signal acquisition devices 150A-150N to enter a standby mode in response to determining that just one of signal acquisition devices 150A-150N has a mismatch.

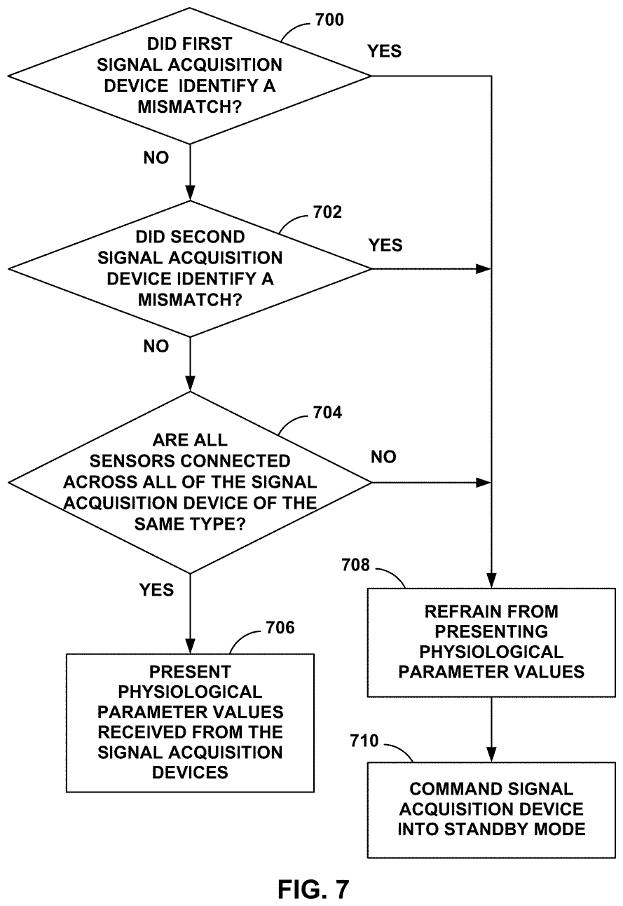

In the example of FIG. 7, processing circuitry 110 determines whether signal acquisition device 150A identified a mismatch among sensors 160A-162A (700). Processing circuitry 110 also determines whether signal acquisition device 150N identified a mismatch among sensors 160N-162N (702). Processing circuitry 110 then determines whether all of sensors 160A-162A and 160N-162N across all of signal acquisition devices 150A-150N are of the same type (704). In response to determining that signal acquisition devices 150A and 150N have not identified a mismatch, and further in response to determining that all of sensors 160A-162A and 160N-162N across all of signal acquisition devices 150A-150N are of the same type, processing circuitry 110 presents physiological parameter values received from signal acquisition devices 150A and 150N (706).

In the example of FIG. 7, responsive to determining that signal acquisition device 150A or 150N has identified a mismatch, or in response to determining that all of sensors 160A-162A and 160N-162N across all of signal acquisition devices 150A-150N are not of the same type, processing circuitry 110 refrains from presenting physiological parameter values from all of signal acquisition devices 150A-150N (708). Processing circuitry 110 commands signal acquisition devices 150A-150N into a standby mode (710). In the standby mode, signal acquisition devices 150A-150N may not calculate physiological parameter values based on sensed signals received from sensors 160A-162A and 160N-162N.

The disclosure contemplates computer-readable storage media comprising instructions to cause a processor to perform any of the functions and techniques described herein. The computer-readable storage media may take the example form of any volatile, non-volatile, magnetic, optical, or electrical media, such as a RAM, ROM, NVRAM, EEPROM, or flash memory. The computer-readable storage media may be referred to as non-transitory. A programmer, such as patient programmer or clinician programmer, or other computing device may also contain a more portable removable memory type to enable easy data transfer or offline data analysis.

The techniques described in this disclosure, including those attributed to device 100, processing circuitry 110, memory 120, display 132, signal acquisition devices 150A-150N, and sensors 160A-160N and 162A-162N, and various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in patient monitors, such as multiparameter patient monitors (MPMs) or other devices, remote servers, or other devices. The term “processor” or “processing circuitry” may generally refer to any of the foregoing logic circuitry, alone or in combination with other logic circuitry, or any other equivalent circuitry.

As used herein, the term “circuitry” refers to an ASIC, an electronic circuit, a processor (shared, dedicated, or group) and memory that execute one or more software or firmware programs, a combinational logic circuit, or other suitable components that provide the described functionality. The term “processing circuitry” refers one or more processors distributed across one or more devices. For example, “processing circuitry” can include a single processor or multiple processors on a device. “Processing circuitry” can also include processors on multiple devices, wherein the operations described herein may be distributed across the processors and devices.

Such hardware, software, firmware may be implemented within the same device or within separate devices to support the various operations and functions described in this disclosure. For example, any of the techniques or processes described herein may be performed within one device or at least partially distributed amongst two or more devices, such as between device 100, processing circuitry 110, memory 120, display 132, signal acquisition devices 150A-150N, and sensors 160A-160N and 162A-162N. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques described in this disclosure may also be embodied or encoded in an article of manufacture including a non-transitory computer-readable storage medium encoded with instructions. Instructions embedded or encoded in an article of manufacture including a non-transitory computer-readable storage medium encoded, may cause one or more programmable processors, or other processors, to implement one or more of the techniques described herein, such as when instructions included or encoded in the non-transitory computer-readable storage medium are executed by the one or more processors. Example non-transitory computer-readable storage media may include RAM, ROM, programmable ROM (PROM), erasable programmable ROM (EPROM), electronically erasable programmable ROM (EEPROM), flash memory, a hard disk, a compact disc ROM (CD-ROM), a floppy disk, a cassette, magnetic media, optical media, or any other computer readable storage devices or tangible computer readable media.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache). Elements of devices and circuitry described herein, including, but not limited to, device 100, processing circuitry 110, memory 120, display 132, signal acquisition devices 150A-150N, and sensors 160A-160N and 162A-162N may be programmed with various forms of software. The one or more processors may be implemented at least in part as, or include, one or more executable applications, application modules, libraries, classes, methods, objects, routines, subroutines, firmware, and/or embedded code, for example.

Various examples of the disclosure have been described. Any combination of the described systems, operations, or functions is contemplated. These and other examples are within the scope of the following claims. The disclosure includes the following examples.

Example 1: A method comprises receiving a first signal from a first signal acquisition device; receiving a second signal from a second signal acquisition device; determining a first type of sensor connected to the first signal acquisition device based on the first signal; determining a second type of sensor connected to the second signal acquisition device based on the second signal; determining that the first type of sensor is different from the second type of sensor; and generating an alert in response to determining that the first type of sensor is different from the second type of sensor connected to the second signal acquisition device.

Example 2: The method of example 1, further comprising commanding the first signal acquisition device to enter a standby mode in response to determining that the first type of sensor connected to the first signal acquisition device is different from the second type of sensor connected to the second signal acquisition device.

Example 3: The method of any one of the preceding examples or any combination thereof, further comprising commanding the second signal acquisition device to enter a standby mode in response to determining that the first type of sensor connected to the first signal acquisition device is different from the second type of sensor connected to the second signal acquisition device.

Example 4: The method of any one of the preceding examples or any combination thereof, wherein the first and second signal acquisition devices do not send data when operating in the standby mode.

Example 5: The method of any one of the preceding examples or any combination thereof, wherein generating the alert comprising presenting, via a display, an indication that the first type of sensor connected to the first signal acquisition device is different from the second type of sensor connected to the second signal acquisition device.

Example 6: The method of any one of the preceding examples or any combination thereof, further comprising refraining from presenting data from the first signal acquisition device in response to determining that the first type of sensor connected to the first signal acquisition device is different from the second type of sensor connected to the second signal acquisition device.

Example 7: The method of any one of the preceding examples or any combination thereof, further comprising refraining from presenting data from the second signal acquisition device in response to determining that the first type of sensor connected to the first signal acquisition device is different from the second type of sensor connected to the second signal acquisition device.

Example 8: A method comprising: receiving a first signal from a first signal acquisition device; receiving a second signal from a second signal acquisition device; determining a mismatch among a second set of sensors connected to the second signal acquisition device based on the second signal; and generating an alert in response to determining the mismatch among the second set of sensors.

Example 9: The method of example 8, further comprising commanding the first signal acquisition device to enter a standby mode in response to determining the mismatch among the second set of sensors.

Example 10: The method of example 8 or example 9, further comprising commanding the second signal acquisition device to enter a standby mode in response to determining the mismatch among the second set of sensors.

Example 11: The method of any one of examples 8-10 or any combination thereof, wherein generating the alert comprising presenting, via a display, an indication of the mismatch among the second set of sensors.

Example 12: The method of any one of examples 8-11 or any combination thereof, further comprising refraining from presenting data from the first signal acquisition device in response to determining the mismatch among the second set of sensors.

Example 13: The method of any one of examples 8-12 or any combination thereof, further comprising refraining from presenting data from the second signal acquisition device in response to determining the mismatch among the second set of sensors.

Example 14: The method of any one of examples 8-13 or any combination thereof, further comprising determining a match among a first set of sensors connected to the first signal acquisition device based on the first signal.

Example 15: A monitoring device comprising: at least two interfaces comprising: a first interface configured to receive a first signal from a first signal acquisition device; and a second interface configured to receive a second signal from a second signal acquisition device; and processing circuitry configured to perform the method of any one of the preceding examples or any combination thereof.

Example 16: The monitoring device of example 15, wherein the processing circuitry is configured to: determine that the first sensor is not different from the second sensor; and determine an algorithm to apply to data received from the first signal acquisition device and from the second signal acquisition device based on a type of the first sensor and the second sensor.

Example 17: A device comprising a computer-readable medium having executable instructions stored thereon, configured to be executable by processing circuitry for causing the processing circuitry to perform the method of any one of examples 1 to 14 or any combination thereof.

Example 18: A system comprising means for performing the method of any one of examples 1 to 14 or any combination thereof.

Example 19: A system comprising: a first sensor and a second sensor; a first signal acquisition device configured to connect to the first sensor; a second signal acquisition device configured to connect to the second sensor; a monitoring device comprising: at least two interfaces comprising: a first interface configured to receive a first signal from the first signal acquisition device indicating a first type of the first sensor connected to the first signal acquisition device; and a second interface configured to receive a second signal from the second signal acquisition device indicating a second type of the second sensor connected to the first signal acquisition device; and processing circuitry configured to perform the method of any one of examples 1 to 14 or any combination thereof.

What is claimed is:

1. A method comprising:
receiving, by processing circuitry, a first signal from a first signal acquisition device;
receiving, by the processing circuitry, a second signal from a second signal acquisition device;
determining, by the processing circuitry, a first type of sensor connected to the first signal acquisition device based on the first signal;
determining, by the processing circuitry, a second type of sensor connected to the second signal acquisition device based on the second signal;
determining, by the processing circuitry, that the first type of sensor is different from the second type of sensor; and
generating a notification in response to determining that the first type of sensor is different from the second type of sensor connected to the second signal acquisition device.

2. The method of claim 1, further comprising commanding, by the processing circuitry, the first signal acquisition device to enter a standby mode in response to determining that the first type of sensor connected to the first signal acquisition device is different from the second type of sensor connected to the second signal acquisition device.

3. The method of claim 2, further comprising commanding, by the processing circuitry, the second signal acquisition device to enter a standby mode in response to determining that the first type of sensor connected to the first signal acquisition device is different from the second type of sensor connected to the second signal acquisition device.

4. The method of claim 3, wherein the first and second acquisition devices are configured to not send data when operating in the respective standby mode.

5. The method of claim 1, wherein generating the notification comprising presenting, via a display, an indication that the first type of sensor connected to the first signal acquisition device is different from the second type of sensor connected to the second signal acquisition device.

6. The method of claim 1, further comprising refraining from presenting data from the first signal acquisition device in response to determining that the first type of sensor connected to the first signal acquisition device is different from the second type of sensor connected to the second signal acquisition device.

7. The method of claim 1, further comprising refraining from presenting data from the second signal acquisition device in response to determining that the first type of sensor connected to the first signal acquisition device is different from the second type of sensor connected to the second signal acquisition device.

8. A system comprising:
at least two interfaces comprising:
a first interface configured to receive a first signal from a first signal acquisition device; and
a second interface configured to receive a second signal from a second signal acquisition device; and
processing circuitry configured to:
determine a first type of sensor connected to the first signal acquisition device based on the first signal,
determine a second type of sensor connected to the second signal acquisition device based on the second signal,
determine that the first type of sensor is different from the second type of sensor, and
generate a notification in response to determining that the first type of sensor is different from the second type of sensor connected to the second signal acquisition device.

9. The system of claim 8, wherein the processing circuitry is configured to:
determine that the first sensor is not different from the second sensor; and
determine an algorithm to apply to data received from the first signal acquisition device and from the second signal acquisition device based on a type of the first sensor and the second sensor.

10. The system of claim 8, wherein the processing circuitry is configured to control the first signal acquisition device to enter a standby mode in response to determining that the first type of sensor connected to the first signal acquisition device is different from the second type of sensor connected to the second signal acquisition device.

11. The system of claim 10, wherein the processing circuitry is configured to control the second signal acquisition device to enter a standby mode in response to determining that the first type of sensor connected to the first signal acquisition device is different from the second type of sensor connected to the second signal acquisition device.

12. The system of claim 11, further comprising the first and second signal acquisition devices, wherein the first and second signal acquisition devices are configured to not send data to the processing circuitry when operating in the respective standby mode.

13. The system of claim 8, further comprising a display, wherein the processing circuitry is configured to generate the notification by at least presenting, via the display, an indication that the first type of sensor connected to the first signal acquisition device is different from the second type of sensor connected to the second signal acquisition device.

14. The system of claim 8, further comprising a display, wherein the processing circuitry is configured to refrain from presenting data from the first signal acquisition device via the display in response to determining that the first type of sensor connected to the first signal acquisition device is different from the second type of sensor connected to the second signal acquisition device.

15. The system of claim 8, further comprising a display, wherein the processing circuitry is configured to refrain from presenting data from the second signal acquisition device in response to determining that the first type of sensor connected to the first signal acquisition device is different from the second type of sensor connected to the second signal acquisition device.

16. The system of claim 8, further comprising:
a first sensor;
a second sensor;
the first signal acquisition device configured to connect to the first sensor; and
the second signal acquisition device configured to connect to the second sensor.

17. A system comprising:
a first interface configured to receive a first signal from a first signal acquisition device; and
a second interface configured to receive a second signal from a second signal acquisition device; and
processing circuitry configured to:
receive the first and second signals;
determine a mismatch among a second set of sensors connected to the second signal acquisition device based on the second signal; and
generate a notification in response to determining the mismatch among the second set of sensors.

18. The system of claim 17, further wherein the processing circuitry is configured to control least one of the first signal acquisition device or the second signal acquisition device to enter a standby mode in response to determining the mismatch among the second set of sensors.

19. The system of claim 17, further comprising the first and second acquisition devices, wherein the first and second acquisition devices are configured to not send data to the processing circuitry when operating in the respective standby mode.

20. The system of claim 17, further comprising a display, wherein the processing circuitry is configured to refrain from presenting data from the first signal acquisition device via the display in response to determining the mismatch among the second set of sensors.
